(19) United States
(12) Patent Application Publication
(10) Pub. No.: US 2025/0000841 A1
(43) Pub. Date: Jan. 2, 2025

(54) Administration of Resiniferatoxin For Treatment of Prostate Cancer

(71) Applicant: Sorrento Therapeutics, Inc., San Diego, CA (US)

(72) Inventors: Henry Hongjun Ji, Rancho Santa Fe, CA (US); Alexis G. Nahama, San Diego, CA (US); Andreas Herrmann, La Jolla, CA (US)

(73) Assignee: Sorrento Therapeutics, Inc., San Diego, CA (US)

(21) Appl. No.: 18/264,973

(22) PCT Filed: Feb. 10, 2022
(86) PCT No.: PCT/US2022/015934

§ 371 Date: Aug. 10, 2023

Related U.S. Application Data
(60) Provisional application No. 63/148,343, filed on Feb. 11, 2021.

(51) Int. Cl. A61K 31/357; A61K 9/08; A61K 47/26; A61P 35/00

(52) U.S. Cl. CPC A61K 31/357; A61K 9/08; A61K 47/26; A61P 35/00

(57) ABSTRACT

Disclosed herein are methods of administering resiniferatoxin (RTX) for treatment of prostate cancer.

### CROSS-REFERENCE TO RELATED APPLICATION

This application claims priority to U.S. provisional patent application No. 63/148,343, filed on Feb. 11, 2021, the content and disclosure of which is incorporated by reference in its entirety for all purposes.

The present disclosure provides methods of treating prostate cancer comprising administering resiniferatoxin (RTX), and resiniferatoxin for use in such methods.

### INTRODUCTION AND SUMMARY

The TRPV-1 receptor is ubiquitously expressed throughout the human body (Velasco et al. Handb Exp Pharmacol. 2015; 231:449-72). Comparing patient biopsy normal versus malignant tissue, shows that TRPV-1 expression is elevated in tumor tissue. Tumor-associated TRPV-1 overexpression can be seen in biopsy immunohistochemistry (FIG. 1A) and Western blot analyses (FIG. 1B).

Resiniferatoxin, herein referred to as RTX, is a TRPV-1 receptor agonist currently used for pain management. Recent studies using RTX illustrated anti-tumor activity of TRPV-1 agonists including RTX and RTX-derivates on human bladder cancer in a rodent xenograft tumor model (Rossi et al. Int J Mol Sci. 2019 Apr. 18; 20 (8)).

In addition, the cytotoxic activity of RTX has been documented in vitro for pancreatic, lung and prostate cancer cells (Ziglioli et al. Acta Biomed. 80 (2009) 13-20; Hartel et al. Gut 55 (2006) 519-528; Hail et al. Apoptosis 8 (2003) 251-262; Athanasiou et al. Biochem. Biophys. Res. Commun. 354 (2007) 50-55).

Resiniferatoxin (RTX) acts as an ultrapotent analog of capsaicin, the pungent principal ingredient of the red pepper. RTX is a tricyclic diterpene isolated from certain species of Eurphorbia. A homovanillyl group is an important structural feature of capsaicin and is the most prominent feature distinguishing resiniferatoxin from typical phorbol-related compounds. Native RTX has the following structure:

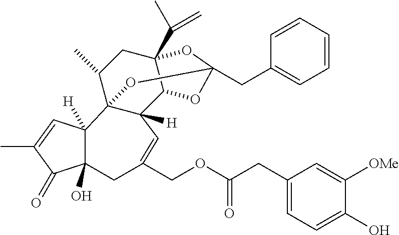 [SMILES: C=C(C)[C@]12C[C@@H](C)[C@@]34OC(Cc5ccccc5)(O[C@@H]1[C@@H]3C=C(COC(=O)Cc1ccc(O)c(OC)c1)C[C@]1(O)C(=O)C(C)=C[C@@H]41)O2]

RTX and analog compounds such as tinyatoxin and other compounds (20-homovanillyl esters of diterpenes such as 12-deoxyphorbol 13-phenylacetate 20-homovanillate and mezerein 20-homovanillate) are described in U.S. Pat. Nos. 4,939,194; 5,021,450; and 5,232,684. Other resiniferatoxin-type phorboid vanilloids have also been identified (Szallasi et al. (1999) Brit. J. Pharmacol. 128:428-434).

RTX is known as a TRPV-1 agonist. TRPV-1, the transient receptor potential cation channel subfamily V member 1 (also known as Vanilloid receptor-1 (VR1)) is a multimeric cation channel prominently expressed in nociceptive primary afferent neurons (Caterina et al. (1997) Nature 389:816-824; Tominaga et al. (1998) Neuron 21:531-543). Activation of TRPV-1 typically occurs at the nerve endings via application of painful heat and is up regulated during certain types of inflammatory stimuli. Activation of TRPV-1 in peripheral tissues by a chemical agonist results in the opening of calcium channels and the transduction of a pain sensation (Szalllasi et al. (1999) Mol. Pharmacol. 56:581-587). However, direct application of certain TRPV-1 agonists to the cell body of a neuron (ganglion) expressing TRPV-1 opens calcium channels and triggers a cascade of events leading to programmed cell death (“apoptosis”) (Karai et al. (2004) J. of Clin. Invest. 113:1344-1352).

Prostate cancer is a common form of cancer and although some treatments are available, it is responsible for 91 deaths per day in the US according to the Prostate Cancer Foundation, which estimates that 1 in 9 US men will be diagnosed with prostate cancer at some point in their lives (see www.pcf.org/about-prostate-cancer/what-is-prostate-cancer/prostate-cancer-survival-rates/). Accordingly, there is a need for improved compositions, methods and uses for treatment of prostate cancer. The present disclosure shows that RTX can be effective against prostate cancer cells, in which TRPV-1 can occur, and aims to meet this need and/or provide other benefits.

Accordingly, the following exemplary embodiments are provided.

Embodiment 1 is a method of treating prostate cancer, comprising administering resiniferatoxin (RTX) to a subject in need of treatment of prostate cancer.

Embodiment 2 is a composition comprising resiniferatoxin (RTX) for use in a method of treating prostate cancer, the method comprising administering RTX to a subject in need of treatment of prostate cancer.

Embodiment 3 is the method or composition for use according to any one of the preceding embodiments, wherein the RTX is administered locally.

Embodiment 4 is the method or composition for use according to any one of the preceding embodiments, wherein the RTX is administered peritumorally.

Embodiment 5 is the method or composition for use according to any one of the preceding embodiments, wherein the subject previously underwent prostate surgery.

Embodiment 6 is the method or composition for use according to any one of the preceding embodiments, wherein the method comprises administering RTX at a concentration of 0.005 mcg/ml-0.01 mcg/ml, 0.01 mcg/ml-0.05 mcg/ml, 0.05 mcg/ml-0.1 mcg/ml, 0.1 mcg/ml-0.15 mcg/ml, 0.15 mcg/ml-0.2 mcg/ml, 0.2 mcg/ml-0.25 mcg/ml, 0.25 mcg/ml-0.3 mcg/ml, 0.30 mcg/ml-0.35 mcg/ml, 0.35 mcg/ml-0.4 mcg/ml, 0.4 mcg/ml-0.45 mcg/ml, 0.45 mcg/ml-0.5 mcg/ml, 0.5 mcg/ml-0.55 mcg/ml, 0.55 mcg/ml-0.6 mcg/ml, 0.6 mcg/ml-0.65 mcg/ml, 0.65 mcg/ml-0.7 mcg/ml, 0.7 mcg/ml-0.75 mcg/ml, 0.75 mcg/ml-0.8 mcg/ml, 0.8 mcg/ml-0.85 mcg/ml, 0.85 mcg/ml-0.9 mcg/ml, 0.9 mcg/ml-0.95 mcg/ml, 0.95 mcg/ml-1.0 mcg/ml, 1.0 mcg/ml-1.1 mcg/ml, or 1.1 mcg/ml-1.2 mcg/ml.

Embodiment 7 is the method or composition for use according to any one of the preceding embodiments, wherein a dose of 0.05 mcg to 0.10 mcg, or 0.10 mcg to 0.15 mcg, or 0.15 mcg to 0.25 mcg, or 0.25 mcg to 0.50 mcg, or 0.50 mcg to 0.75 mcg, or 0.75 mcg to 1.0 mcg, or 1.0 mcg to 1.1 mcg, or 1.1 mcg to 1.5 mcg of RTX is administered.

Embodiment 8 is the method or composition for use according to embodiment 7, wherein the RTX is administered at a dose of at least about 0.1 mcg.

Embodiment 9 is the method or composition for use according to embodiment 7, wherein the RTX is administered at a dose of at least about 0.5 mcg.

Embodiment 10 is the method or composition for use according to embodiment 7, wherein the RTX is administered at a dose of at least about 1.0 mcg.

Embodiment 11 is the method or composition for use of any one of the preceding embodiments, wherein the RTX is administered in one dose.

Embodiment 12 is the method or composition for use of any one of the preceding embodiments, wherein the RTX is administered in repeated doses.

Embodiment 13 is the method or composition for use of any one of the preceding embodiments, wherein the RTX is administered daily.

Embodiment 14 is the method or composition for use of any one of the preceding embodiments, wherein the RTX is administered every other day.

Embodiment 15 is the method or composition for use of any one of the preceding embodiments, wherein the subject is a mammal.

Embodiment 16 is the method or composition for use of embodiment 15, wherein the mammal is a human.

Embodiment 17 is the method or composition for use according to any one of the preceding embodiments, wherein the prostate cancer is prostate adenocarcinoma.

Embodiment 18 is the method or composition for use according to any one of the preceding embodiments, wherein the method comprises administering a pharmaceutical formulation comprising the RTX and a pharmaceutically acceptable carrier.

Embodiment 19 is the method or composition for use of embodiment 18, wherein the pharmaceutically acceptable carrier comprises water.

Embodiment 20 is the method or composition for use of embodiment 18 or 19, wherein the pharmaceutically acceptable carrier comprises polysorbate 80.

Embodiment 21 is the method or composition for use of any one of embodiments 18-20, wherein the pharmaceutically acceptable carrier comprises a buffer, optionally wherein the buffer is phosphate buffer and/or the pH of the formulation is about 7.0-7.5 or about 7.2.

### BRIEF DESCRIPTION OF THE DRAWINGS

FIGS. 1A and 1B show that RTX receptor TRPV-1 is overexpressed in prostate carcinoma. FIG. 1A shows broad expression of TRPV-1 throughout the human body. Vertical axis units are fold increase in mRNA expression. FIG. 1B shows TRPV-1 overexpression in prostate carcinoma as compared to adjacent normal tissue assessed from patient biopsies. Scale, 50 μm.

FIGS. 2A, 2B, and 2C show RTX inhibits proliferation of TRPV-1⁺ prostate carcinoma cell lines. In FIG. 2A-B Human prostate carcinoma cell lines DU145 and LNCaP are shown to express TRPV-1 by flow cytometry. Horizontal axis units in FIG. 2A-B are arbitrary fluorescence units. FIG. 2C shows RTX treatment decreases prostate carcinoma cell lines proliferation in a dose-dependent manner.

FIGS. 3A, 3B and 3C show RTX inhibits prostate carcinoma progression in a cytostatic manner. FIG. 3A shows tumor volume over time for human prostate cancer DU145 cells engrafted s.c. in mice to assess anti-tumoral cytostatic activity by RTX. RTX was administered locally (peritumorally) every other day at the indicated dose starting at 28 days after engraftment. The cytostatic activity appeared to be dose-independent in the tested range of 0.1-1 μg/dose. FIG. 3B shows RTX administration resulted in loss of tumor tissue integrity (upper row), significantly reduced CD31⁺ tumor vasculature (middle row), and significantly increased cleaved caspase 3⁺ tumor cell apoptosis (lower row), as analyzed by confocal microscopy of tumor tissue sections. Scale, 100 μm. CD31+ blood vessel length and cleaved caspase 3 levels (expressed as median fluorescence intensity, MFI) are quantified in FIG. 3C.

FIGS. 4A, 4B and 4C show that RTX treatment does not induce Cytokine Release Syndrome but reduces IL-6 expression. FIG. 4A shows Tumor (TM) homogenates (WCL: whole cell lysate) isolated from tumors treated as indicated used to assess cytokine expression by a cytokine array tailored for assessment of inflammatory cytokines. Reduced IL-6 expression in a dose-dependent manner is shown magnified (FIG. 4A). FIG. 4C shows the contents of each location of the array of FIG. 4A.

### DETAILED DESCRIPTION OF CERTAIN EMBODIMENTS

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### A. Definitions

“Prostate cancer” refers to any condition in which malignant cells are present in the prostate.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### B. Exemplary Methods and Compositions for Use

Provided herein are methods for treating prostate cancer, comprising administering resiniferatoxin (RTX) to a subject in need of treatment of prostate cancer. Also provided are compositions comprising RTX for use in a method of treating prostate cancer, the method comprising administering RTX to a subject in need of treatment of prostate cancer. In some embodiments the subject previously underwent prostate surgery.

In some embodiments, the RTX is administered locally. In some embodiments the RTX is administered peritumorally.

The compositions and methods described herein are for use with any subject in whom RTX is effective, e.g., able to bind and activate TRPV-1 or a homolog thereof, and who is in need of treatment for prostate cancer. In some embodiments, the subject is a mammal. In some embodiments, the mammal is a human. In some embodiments, the mammal is a cat. In some embodiments, the mammal is a dog.

### 1. Dosage

In some embodiments, the RTX is administered (e.g., peritumorally) at a dose of 0.05 mcg to 0.10 mcg, or 0.10 mcg to 0.15 mcg, or 0.15 mcg to 0.25 mcg, or 0.25 mcg to 0.50 mcg, or 0.50 mcg to 0.75 mcg, or 0.75 mcg to 1.0 mcg, or 1.0 mcg to 1.1 meg, or 1.1 mcg to 1.5 mcg. In some embodiments, the RTX is administered (e.g., systemically) at a dose of at least about 0.1 mcg/kg, such as 0.1 mcg/kg-0.2 mcg/kg, 0.2 mcg/kg-0.3 mcg/kg, 0.3 mcg/kg-0.4 mcg/kg, 0.4 mcg/kg-0.5 mcg/kg, 0.5 mcg/kg-0.6 mcg/kg, 0.6 mcg/kg-0.7 mcg/kg, 0.7 mcg/kg-0.8 mcg/kg, 0.8 mcg/kg-0.9 mcg/kg, 0.9 mcg/kg-1 mcg/kg, 1 mcg/kg-1.2 mcg/kg, 1.2 mcg/kg-1.4 mcg/kg, 1.4 mcg/kg-1.6 mcg/kg, 1.6 mcg/kg-1.8 mcg/kg, 1.8 mcg/kg-2.0 mcg/kg, 2.0 mcg/kg-2.2 mcg/kg, 2.2 mcg/kg-2.4 mcg/kg, 2.4 mcg/kg-2.6 mcg/kg, 2.6 mcg/kg-2.8 mcg/kg, 2.8 mcg/kg-3.0 mcg/kg, 3.0 mcg/kg-3.2 mcg/kg, 3.2 mcg/kg-3.4 mcg/kg, 3.4 mcg/kg-3.6 mcg/kg, 3.6 mcg/kg-3.8 mcg/kg, 4.0 mcg/kg-4.2 mcg/kg, 4.2 mcg/kg-4.4 mcg/kg, 4.4 mcg/kg-4.6 mcg/kg, 4.6 mcg/kg-4.8 mcg/kg, 4.8 mcg/kg-5.0 mcg/kg, 5.0 mcg/kg-5.2 mcg/kg, 5.2 mcg/kg-5.4 mcg/kg, 5.4 mcg/kg-5.6 mcg/kg, 5.6 mcg/kg-5.8 mcg/kg, or 5.8 mcg/kg-6.0 mcg/kg.

In some embodiments, the RTX is administered at a dose of about 0.1 mcg, or about 0.5 mcg, or about 1.0 mcg.

In some embodiments, the RTX is delivered in a composition having a volume of 0.2 ml-0.5 ml, 0.5 ml-1.0 ml, 1 ml-10 ml, 20 ml-30 ml, 30 ml-40 ml, 40 ml-50 ml, 50 ml-60 ml, 60 ml-70 ml, 70 ml-80 ml, 80 ml-90 ml, or 90 ml-100 ml.

In some embodiments, the RTX is administered in one dose. In some embodiments, the RTX is administered in repeated doses. In some embodiments, the RTX is administered in 1, 2, 3, 4, or 5 doses.

In some embodiments, the RTX is administered daily. In some embodiments, the RTX is administered every other day. In some embodiments, the RTX is administered weekly.

### 2. Formulations

Multiple examples of formulations of RTX are available in the literature. See, e.g., Ueda et al. (2008) J. of Cardiovasc. Pharmacol. 51:513-520, and US 2015/0190509 A1. Any suitable formulation of RTX for administration may be used. In some embodiments, RTX is prepared for administration by dilution in saline.

In some embodiments, the RTX, which may be at the dosages discussed above, is administered with a pharmaceutically acceptable carrier. In some embodiments, the pharmaceutically acceptable carrier comprises water. In some embodiments, the pharmaceutically acceptable carrier comprises saline. In some embodiments, the pharmaceutically acceptable carrier comprises polysorbate 80. In some embodiments, the pharmaceutically acceptable carrier comprises polyethylene glycol. In some embodiments, the pharmaceutically acceptable carrier comprises sugar or sugar alcohol. In some embodiments, the pharmaceutically acceptable carrier comprises mannitol. In some embodiments, the pharmaceutically acceptable carrier comprises dextrose. In some embodiments, the pharmaceutically acceptable carrier comprises a pharmaceutically acceptable buffer. In some embodiments, the pharmaceutically acceptable carrier comprises a phosphate buffer. In some embodiments, the pharmaceutically acceptable carrier comprises a pharmaceutically acceptable salt. In some embodiments, the pharmaceutically acceptable carrier comprises NaCl. In some embodiments, the pharmaceutically acceptable carrier comprises an organic solvent such as ethanol or DMSO, e.g., as a minority or residual component used as an aid in dissolving RTX before dilution in a primarily aqueous composition.

In some embodiments, the concentration of RTX in the formulation may be any suitable value for delivery of the intended dose. In some embodiments, the concentration of RTX in the formulation may be any suitable value for storage and may be diluted to obtain a concentration that is suitable for delivery of the intended dose.

In some embodiments, the concentration of RTX in the pharmaceutical formulation is in the range of 0.1 to 300 mcg/ml.

In some embodiments, the concentration of RTX in the pharmaceutical formulation is in the range of 0.1-1 mcg/ml, 1-5 mcg/ml, 5-10 mcg/ml, 10-20 mcg/ml, 10-30 mcg/ml, 20-30 mcg/ml, 20-50 mcg/ml, 50-100 mcg/ml, 100-150 mcg/ml, 150-200 mcg/ml, 200-250 mcg/ml, or 250-300 mcg/ml. In some embodiments, the concentration of RTX in the pharmaceutical formulation is 0.005 mcg/ml-0.01 mcg/ml, 0.01 mcg/ml-0.05 mcg/ml, 0.05 mcg/ml-0.1 mcg/ml, 0.1 mcg/ml-0.15 mcg/ml, 0.15 mcg/ml-0.2 mcg/ml, 0.2 mcg/ml-0.25 mcg/ml, 0.25 mcg/ml-0.3 mcg/ml, 0.30 mcg/ml-0.35 mcg/ml, 0.35 mcg/ml-0.4 mcg/ml, 0.4 mcg/ml-0.45 mcg/ml, 0.45 mcg/ml-0.5 mcg/ml, 0.5 mcg/ml-0.55 mcg/ml, 0.55 mcg/ml-0.6 mcg/ml, 0.6 mcg/ml-0.65 mcg/ml, 0.65 mcg/ml-0.7 mcg/ml, 0.7 mcg/ml-0.75 mcg/ml, 0.75 mcg/ml-0.8 mcg/ml, 0.8 mcg/ml-0.85 mcg/ml, 0.85 mcg/ml-0.9 mcg/ml, 0.9 mcg/ml-0.95 mcg/ml, 0.95 mcg/ml-1.0 mcg/ml, 1.0 mcg/ml-1.1 mcg/ml, or 1.1 mcg/ml-1.2 mcg/ml.

The formulation may have any pH suitable for administration. In some embodiments, the pharmaceutical formulation comprising the RTX and a pharmaceutically acceptable carrier has a pH in the range of 6 to 7.6. In some embodiments, the pharmaceutical formulation comprising the RTX and a pharmaceutically acceptable carrier has a pH in the range of 6 to 6.4, 6.3 to 6.7, 6.4 to 6.8, 6.8 to 7.2, 7 to 7.4, or 7.2 to 7.6. In some embodiments, the pharmaceutical formulation comprising the RTX and a pharmaceutically acceptable carrier has a pH of 6.5 or 7.2.

In some embodiments, the formulation comprises polysorbate 80. In some embodiments, the concentration of polysorbate 80 is 0.03-7% w/v. In some embodiments, the concentration of polysorbate 80 is 2-4% w/v. In some embodiments, the concentration of polysorbate 80 is 3% w/v. The formulation may further comprise a buffer, such as phosphate buffer (e.g., sodium phosphate buffer). In some embodiments, the concentration of phosphate buffer is 10-50 mM. In some embodiments, the concentration of phosphate buffer is 10-30 mM. In some embodiments, the concentration of phosphate buffer is 10 mM. In some embodiments, the concentration of phosphate buffer is 30 mM. The formulation may have a pH in the range of 7-7.5, such as about 7.2. In some embodiments, in any of the foregoing formulations, the concentration of RTX may be 10-30 mcg/ml, such as 10 mcg/ml or 25 mcg/ml. In some embodiments, the formulation further comprises phosphate buffer, e.g., at a concentration and pH shown for phosphate buffer in Table 1. In some embodiments, the formulation further comprises NaCl, e.g., at a concentration shown for NaCl in Table 1. When both are present, the phosphate buffer and NaCl may be (but are not necessarily) present at a combination of concentrations and phosphate buffer pH shown for an individual formulation.

Exemplary formulations of RTX are shown in the following table.

TABLE 1
Exemplary RTX Solution Formulations

| Formula- | | | |
|---|---|---|---|
| tion | | Component | |
| Number | Formulation Components | Concentration | |
| 1 | RTX | 200 | mcg/mL |
| | Polysorbate 80 | 7.0% | w/v |
| | Dextrose | 0.8% | w/v |
| | 30 mM Phosphate Buffer w/0.44% NaCl | 30 mM, pH 7.2 | |
| 2 | RTX | 200 | mcg/mL |
| | Polyethylene Glycol 300 | 3.0% | v/v |
| | Polysorbate 80 | 0.1% | w/v |
| | Dextrose | 0.8% | w/v |
| | 10 mM Phosphate Buffer w/0.73% NaCl | 10 mM, pH 6.5 | |
| 3 | RTX | 200 | mcg/mL |
| | Polyethylene Glycol 300 | 30.0% | v/v |
| | Polysorbate 80 | 1.0% | w/v |
| | 10 mM Phosphate Buffer w/0.86% NaCl | 10 mM, pH 6.5 | |
| 4 | RTX | 200 | mcg/mL |
| | Polyethylene Glycol 300 | 30.0% | v/v |
| | Polysorbate 80 | 0.04% | w/v |
| | 10 mM Phosphate Buffer w/0.88% NaCl | 10 mM, pH 6.5 | |
| 5 | RTX | 200 | mcg/mL |
| | Polysorbate 80 | 3.0% | w/v |
| | Dextrose | 0.8% | w/v |
| | 30 mM Phosphate Buffer w/0.54% NaCl | 30 mM, pH 7.2 | |
| 6 | RTX | 200 | mcg/mL |
| | Polysorbate 80 | 3.0% | w/v |
| | Mannitol | 0.8% | w/v |
| | 30 mM Phosphate Buffer w/0.54% NaCl | 30 mM, pH 7.2 | |
| 7 | RTX | 200 | mcg/mL |
| | Polysorbate 80 | 7.0% | w/v |
| | Mannitol | 0.8% | w/v |
| | 30 mM Phosphate Buffer w/0.45% NaCl | 30 mM, pH 7.2 | |
| 8 | RTX | 200 | mcg/mL |
| | Polyethylene Glycol 300 | 3.0% | v/v |
| | Polysorbate 80 | 0.1% | w/v |
| | Mannitol | 0.8% | w/v |
| | 10 mM Phosphate Buffer w/0.74% NaCl | 10 mM, pH 6.5 | |
| 9 | RTX | 200 | mcg/mL |
| | Polyethylene Glycol 300 | 3.0% | v/v |
| | Polysorbate 80 | 0.1% | w/v |
| | Dextrose | 3.0% | w/v |
| | 10 mM Phosphate Buffer w/0.34% NaCl | 10 mM, pH 6.5 | |
| 10 | RTX | 200 | mcg/mL |
| | Polyethylene Glycol 300 | 3.0% | v/v |
| | Polysorbate 80 | 0.1% | w/v |
| | Mannitol | 3.0% | w/v |
| | 10 mM Phosphate Buffer w/0.36% NaCl | 10 mM, pH 6.5 | |
| 11 | RTX | 200 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 12 | RTX | 200 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 13 | RTX | 25 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 14 | RTX | 25 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 15 | RTX | 100 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 16 | RTX | 200 | mcg/mL |
| | Polysorbate 80 | 7.0% | w/v |
| | Dextrose | 5.0% | w/v |

| | |
|---|---|
| 30 mM Phosphate Buffer w/0.54% NaCl | 30 mM, pH 7.2 |

In some embodiments, formulations in Table 1 include dextrose. In embodiments, the concentration of dextrose is 0.05-5% w/v. In some embodiments, the concentration of dextrose is 0.8-5% w/v. In some embodiments, the concentration of dextrose is 0.05% w/v. In some embodiments, the concentration of dextrose is 0.8% w/v. In some embodiments, the concentration of dextrose is 3.0% w/v. In some embodiments, the concentration of dextrose is 5.0% w/v.

In some embodiments, formulations in Table 1 include mannitol. In some embodiments, the concentration of mannitol is 0.8-3.0% w/v. In some embodiments, the concentration of mannitol is 0.8% w/v. In some embodiments, the concentration of mannitol is 3.0% w/v.

In some embodiments, the dextrose or mannitol is omitted from a formulation shown in Table 1.

In some embodiments, the concentration of RTX in a formulation shown in Table 1 is adjusted to any of the RTX concentrations or concentration ranges disclosed herein. For example, in some embodiments, the concentration of RTX in a formulation shown in Table 1 is adjusted to 0.3-200 mcg/ml. In some embodiments, the concentration of RTX in a formulation shown in Table 1 is 200 mcg/ml. In some embodiments, the concentration of RTX in a formulation shown in Table 1 is 0.3-100 mcg/ml. In some embodiments, the concentration of RTX in a formulation shown in Table 1 is 100 mcg/ml. In some embodiments, the concentration of RTX in a formulation shown in Table 1 is adjusted to 0.3-50 mcg/ml. In some embodiments, the concentration of RTX in a formulation shown in Table 1 is 25 mcg/ml. As another example, in some embodiments, the concentration of RTX in a formulation shown in Table 1 is adjusted to 0.3-15 mcg/ml. As another example, in some embodiments, the concentration of RTX in a formulation shown in Table 1 is adjusted to 0.5-10 mcg/ml. As another example, in some embodiments, the concentration of RTX in a formulation shown in Table 1 is adjusted to 0.6-1.5 mcg/ml. The dextrose or mannitol is omitted from any such formulation having an adjusted RTX concentration.

The formulations in Table 1 may be prepared according to the following exemplary methods, which are provided for formulations 3 and 5 but may be adapted to the other formulations by one skilled in the art. Formulation 3 may be made by adding 46 mg sodium phosphate monobasic monohydrate, 94.7 mg sodium phosphate dibasic anhydrous, and 860 mg NaCl to a 100 ml volumetric flask. 50 ml of water for injection (WFI) is added to dissolve the components in the flask, followed by addition of 1.0 g of polysorbate 80, to form the aqueous component. 20 mg of RTX is added to the aqueous component in the volumetric flask, and pH is adjusted with hydrochloric acid/sodium hydroxide to 7.2. Then 30 mL of PEG 300 is added and the solution is sonicated to dissolve the solids. It should be noted that RTX will sometimes precipitate at the interface of aqueous solution and PEG initially, but will go back into solution upon sonication. The full mixture in the flask is diluted to volume (100.00 ml) with water (WFI) and this is mixed by an inversion process. The full formulation is filtered through a 0.2 μm polytetrafluoroethylene (PTFE) filter.

Formulation 5 may be made by adding 138 mg sodium phosphate monobasic monohydrate, 284.1 mg sodium phosphate dibasic anhydrous, and 540 mg NaCl to a 100 ml volumetric flask. 50 ml of water for injection (WFI) is added to dissolve the components in the flask, followed by addition of 3.0 g of polysorbate 80, and 800 mg of dextrose to form the aqueous component. 20 mg of RTX is added the aqueous component in the volumetric flask, and pH is adjusted with hydrochloric acid/sodium hydroxide to 7.2. The solution is then sonicated to dissolve all the solids. (Alternatively, the RTX may be initially dissolved in a small volume of ethanol or DMSO, and this solution may then be added to the aqueous component.) The full mixture in the flask is diluted to volume (100.00 ml) with water (WFI) and this is mixed by an inversion process. The full formulation is filtered through a 0.2 μm PTFE filter.

A formulation according to Formulation 11 is prepared using 200 mcg RTX, 300 mcg Polysorbate 80 (using commercially-available polysorbate 80); 5.4 mg of sodium chloride, 500 mcg of dextrose, 1.38 mg sodium phosphate monobasic monohydrate, 2.84 mg sodium phosphate dibasic anhydrous, and water (WFI) to 1 mL, then pH is adjusted with hydrochloric acid/sodium hydroxide to 7.2. As noted above, the dextrose may be omitted.

A formulation according to Formulation 13 is prepared using 25 mcg RTX, 30 mg Polysorbate 80 (using commercially-available polysorbate 80); 5.4 mg of sodium chloride, 50 mg of dextrose, 1.38 mg sodium phosphate monobasic monohydrate, 2.84 mg sodium phosphate dibasic anhydrous, water (WFI) to 1 mL, then pH is adjusted with hydrochloric acid/sodium hydroxide to 7.2. As noted above, the dextrose may be omitted.

Further details on techniques for formulation and administration may be found in Gennaro, A., Ed., Remington's Pharmaceutical Sciences, 18th Ed. (1990) (Mack Publishing Co., Easton, Pa.).

### EXAMPLES

### A. Anti-Prostate-Carcinoma Efficacy of Resiniferatoxin in Prostate Cancer Cell Lines

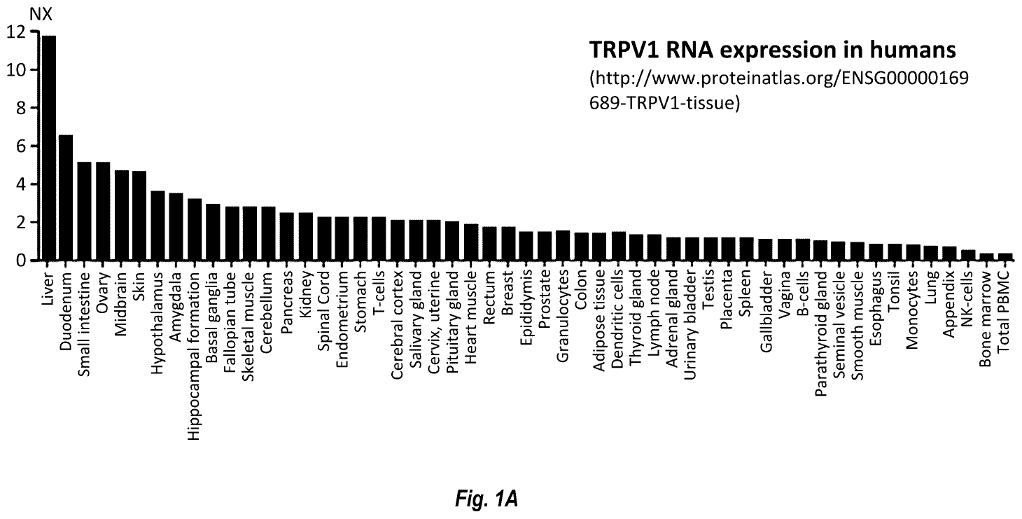

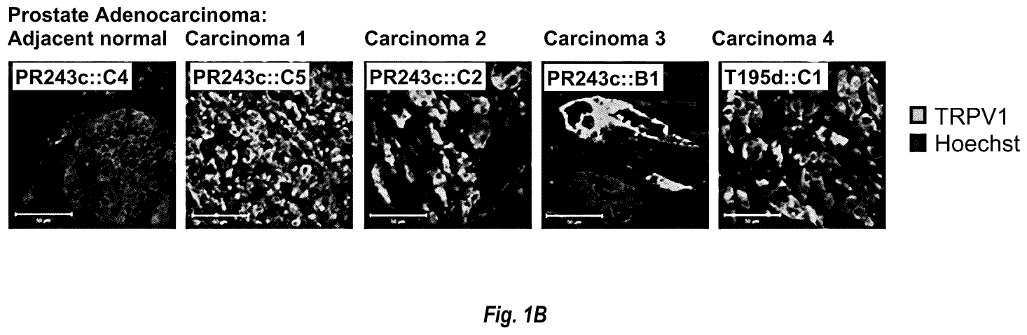

Although TRPV-1 is ubiquitously expressed throughout the human body (FIG. 1A), it was observed that TRPV-1 was overexpressed in prostate carcinoma (PCa) (FIG. 1B). Specifically, immunohistochemically stained patient biopsies of prostate adenocarcinoma analyzed by confocal laser scanning microscopy revealed TRPV-1 overexpression in prostate cancer.

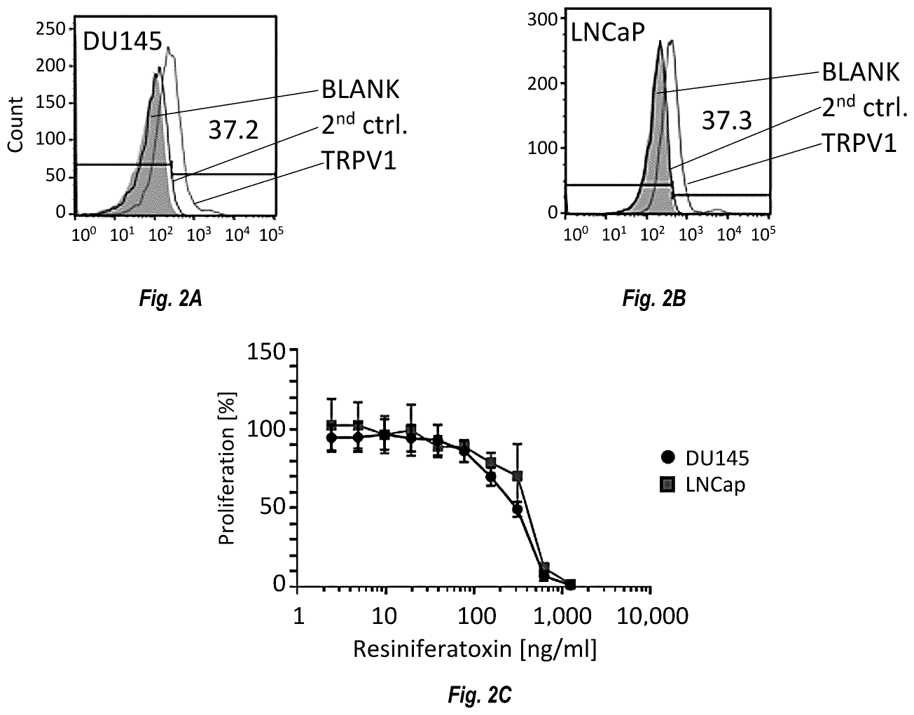

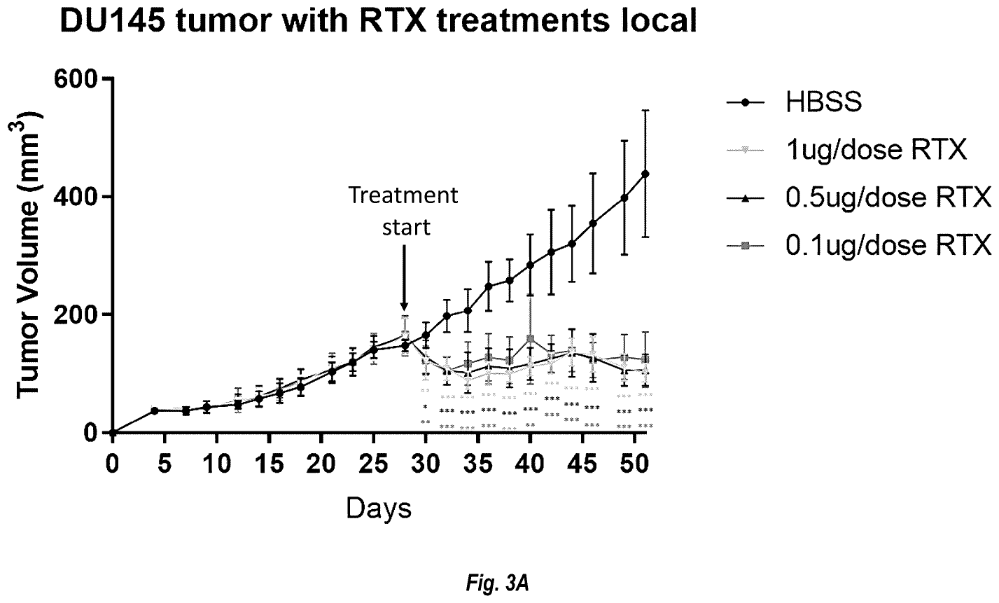

To assess RTX anti-PCa activity, TRPV-1 expression by human prostate cancer cell lines DU145 and LNCaP was validated by flow cytometry (FIGS. 2A-B), with both responding to RTX treatment and showing dose-dependent decreases in proliferation (FIG. 2C). Specifically, TRPV-1 overexpression by human prostate cancer cell lines DU145 and LNCaP was demonstrated by flow cytometry (FIG. 2A, 2B) and both cell lines responded to RTX treatment in a dose-dependent manner by reduced cell division and/or cancer cell death as shown by proliferation assay (FIG. 2C). Cells were cultured in media for in the presence of varying amounts of RTX as shown in FIG. 2C. Proliferation measurements were normalized to an untreated control.

It was observed that prostate adenocarcinoma potentially has increased sensitivity to RTX by overexpression of its receptor TRPV-1. Furthermore, reduced prostate cancer cell proliferation upon low-dose treatment suggested that RTX exerts a robust antitumoral potential.

### B. Anti-Prostate-Carcinoma Efficacy of Resiniferatoxin in a Mouse Xenograft Tumor Model

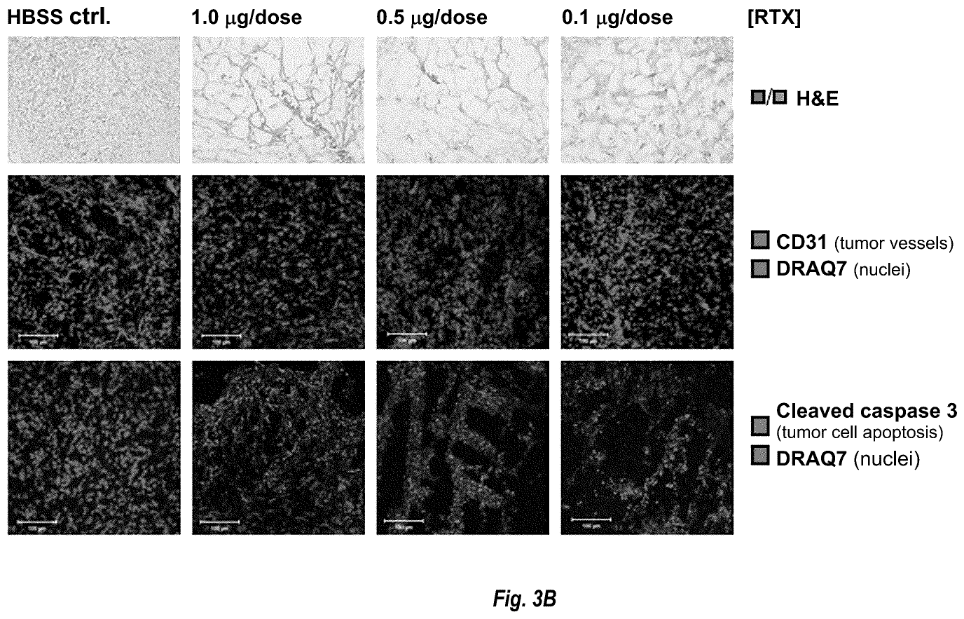

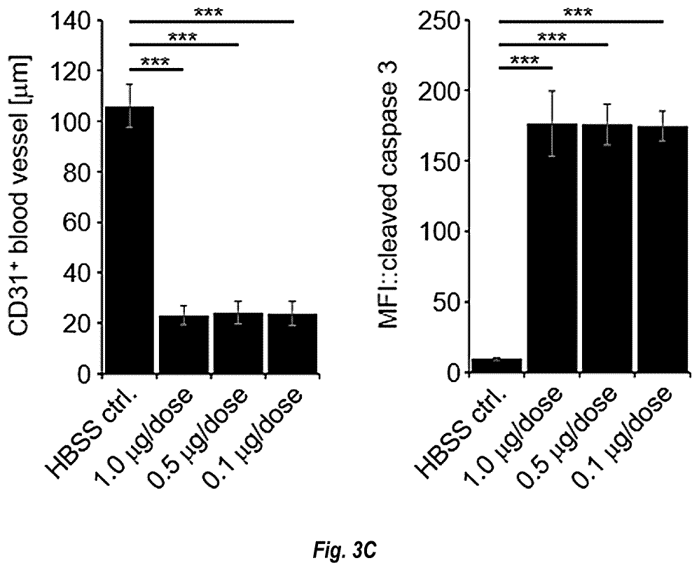

In a mouse xenograft tumor model engrafting human DU145 prostate carcinoma cells s.c., cytostatic efficacy by local administration of RTX was demonstrated. Notably, RTX doses as low as 0.1 μg/dose administered locally every other day exert a potent cytostatic efficacy in a dose-independent manner over dosages of 0.1, 0.5, and 1 μg. (FIG. 3A).

Dissected tumor tissues were assessed by H&E staining and by immunofluorescence for CD31 (tumor vessel marker), DRAQ7 (nuclear marker), and cleaved caspase 3 (apoptotic cell maker). RTX administration at 1.0 μg/dose, 0.5 μg/dose, and 0.1 μg/dose resulted in a loss of tumor tissue integrity indicative of tumor cell apoptosis and necrosis and as evidenced by characteristic morphological changes in the tissue consistent with cell death, including the appearance of large substantially unstained regions relative to the control. (FIG. 3B, upper row). Moreover, treatment of human prostate tumors with RTX exerts anti-tumor vasculature efficacy as shown by decreased contiguous CD31+ stained structures in the tumor tissue showing a loss of long track blood vessel morphology. (FIG. 3B, middle row). Furthermore, tumor cell apoptosis increased upon RTX administration as evidenced by increases in cleaved caspase 3 reporter staining. (FIG. 3B, lower row; Quantifications shown in FIG. 3C).

C. Decreased IL-6 Production Upon Treatment with Resiniferatoxin

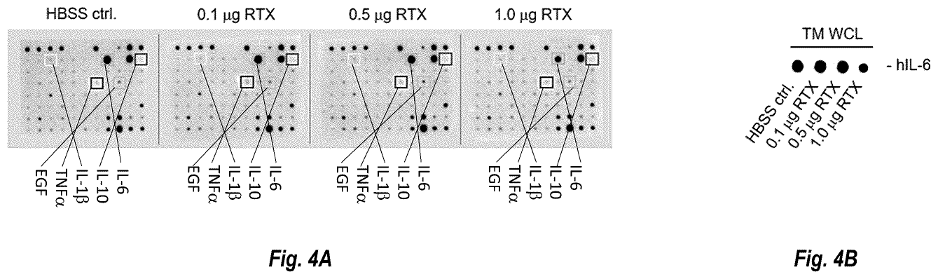

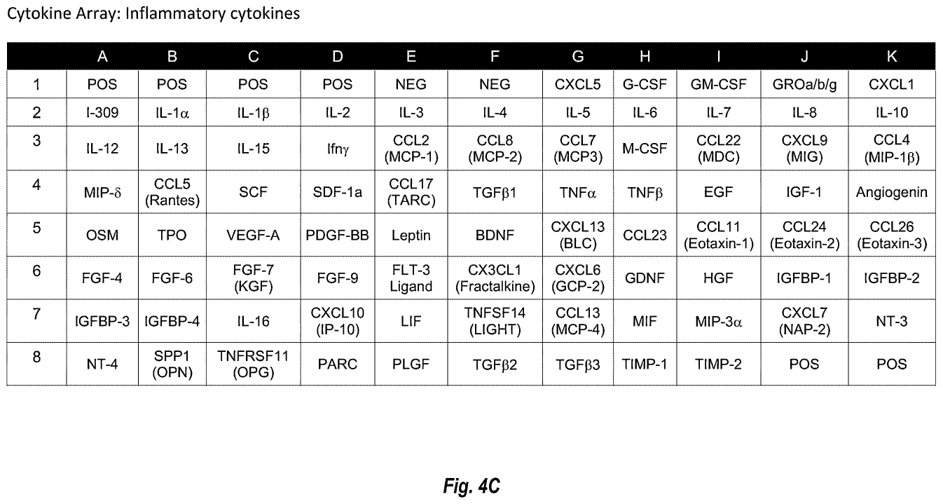

Some studies of RTX have reported a potential to induce neurologic inflammatory responses. In this study, however, cytokine expression was not noticeably elevated in that there was no indication of Cytokine Release Syndrome (CRS) upon RTX treatment. Tumor tissue homogenate samples from mouse xenograft tumor models described in Example B were taken at necroptic endpoint and analyzed for tumor associated inflammatory cytokine levels using a cytokine array (FIG. 4A).

Interleukin-6 (IL-6), considered one of the major mediators of tumor inflammation, shows reduced expression upon treatment with RTX at higher doses (FIG. 4A-C). Decreased IL-6 production upon RTX treatment is indicative of reduced tumor inflammatory responses.

### D. Discussion and Summary of Results

RTX administration has shown promising efficacy in pain management of patients battling advanced tumors. It has now been demonstrated that RTX exerts direct anti-tumoral activity in a human prostate carcinoma xenograft tumor model. RTX treatment significantly reduced tumor growth progression and growth in a dose-independent manner. Moreover, RTX treatment showed cytostatic efficacy in vivo.

RTX is known to engage with its cognate receptor TRPV-1 on the surface of cells, thereby, inducing Ca2+ cation influx. Ca2+ cation uptake by cancer cells is known to promote apoptotic cell death. However, Ca2+ cation influx is a hallmark of immediate early activation of the adaptive immune response. Thus, RTX induced Ca2+ cation influx may contribute to a desired anti-tumoral T cell response, exerted by further matured CD8 T cells into IFNg+Gr.B+ cytotoxic T lymphocytes (CTLs).

RTX administration did not induce CRS-related cytokine expression, and in contrast, RTX treatment drastically reduced IL-6 expression. IL-6 is one of the major mediators of tumor inflammation associated with poor prognosis.

IL-6 expression is transcriptionally controlled by NFκB signaling triggered by a broad range of extracellular ligands, often by signals related to pathogens or stress, such as LPS, TNFα, IL-1β and numerous Toll-like receptor (TLR) signaling pathways merging into NFκB activation. However, reduced IL-6 production indicates dampened NFκB signaling indicative of reduced tumor tissue inflammation. Moreover, decreased IL-6 levels found in tumor tissue is expected to lower IL-6 induced JAK/STAT3 activity, which represents a key node in the molecular regulation of inflammation.

Although IL-6/IFNγ and their mediators STAT3/STAT1, respectively, have been shown to be counterbalanced (Costa-Pereira, et al., PNAS Jun. 11, 2002 99 (12) 8043-8047), RTX treatment did not induce elevated expression of IFNγ, a result that indicates an antitumoral adaptive immune response. Hence, in addition to the direct anti-tumoral activity exerted by RTX demonstrated herein, RTX may trigger an adaptive immune response mounting a potent antitumoral efficacy.

What is claimed is:

1. A method of treating prostate cancer, comprising administering resiniferatoxin (RTX) to a subject in need of treatment of prostate cancer.

2. A composition comprising resiniferatoxin (RTX) for use in a method of treating prostate cancer, the method comprising administering RTX to a subject in need of treatment of prostate cancer.

3. The method or composition for use according to any one of the preceding claims, wherein the RTX is administered locally.

4. The method or composition for use according to any one of the preceding claims, wherein the RTX is administered peritumorally.

5. The method or composition for use according to any one of the preceding claims, wherein the subject previously underwent prostate surgery.

6. The method or composition for use according to any one of the preceding claims, wherein the method comprises administering RTX at a concentration of 0.005 mcg/ml-0.01 mcg/ml, 0.01 mcg/ml-0.05 mcg/ml, 0.05 mcg/ml-0.1 mcg/ml, 0.1 mcg/ml-0.15 mcg/ml, 0.15 mcg/ml-0.2 mcg/ml, 0.2 mcg/ml-0.25 mcg/ml, 0.25 mcg/ml-0.3 mcg/ml, 0.30 mcg/ml-0.35 mcg/ml, 0.35 mcg/ml-0.4 mcg/ml, 0.4 mcg/ml-0.45 mcg/ml, 0.45 mcg/ml-0.5 mcg/ml, 0.5 mcg/ml-0.55 mcg/ml, 0.55 mcg/ml-0.6 mcg/ml, 0.6 mcg/ml-0.65 mcg/ml, 0.65 mcg/ml-0.7 mcg/ml, 0.7 mcg/ml-0.75 mcg/ml, 0.75 mcg/ml-0.8 mcg/ml, 0.8 mcg/ml-0.85 mcg/ml, 0.85 mcg/ml-0.9 mcg/ml, 0.9 mcg/ml-0.95 mcg/ml, 0.95 mcg/ml-1.0 mcg/ml, 1.0 mcg/ml-1.1 mcg/ml, or 1.1 mcg/ml-1.2 mcg/ml.

7. The method or composition for use according to any one of the preceding claims, wherein a dose of 0.05 mcg to 0.10 mcg, or 0.10 mcg to 0.15 mcg, or 0.15 mcg to 0.25 mcg, or 0.25 mcg to 0.50 mcg, or 0.50 mcg to 0.75 mcg, or 0.75 mcg to 1.0 mcg, or 1.0 mcg to 1.1 mcg, or 1.1 mcg to 1.5 mcg of RTX is administered.

8. The method or composition for use according to claim 7, wherein the RTX is administered at a dose of about 0.1 mcg.

9. The method or composition for use according to claim 7, wherein the RTX is administered at a dose of about 0.5 mcg.

10. The method or composition for use according to claim 7, wherein the RTX is administered at a dose of about 1.0 mcg.

11. The method or composition for use of any one of the preceding claims, wherein the RTX is administered in one dose.

12. The method or composition for use of any one of the preceding claims, wherein the RTX is administered in repeated doses.

13. The method or composition for use of any one of the preceding claims, wherein the RTX is administered daily.

14. The method or composition for use of any one of the preceding claims, wherein the RTX is administered every other day.

15. The method or composition for use of any one of the preceding claims, wherein the subject is a mammal.

16. The method or composition for use of claim 15, wherein the mammal is a human.

17. The method or composition for use according to any one of the preceding claims, wherein the prostate cancer is prostate adenocarcinoma.

18. The method or composition for use according to any one of the preceding claims, wherein the method comprises administering a pharmaceutical formulation comprising the RTX and a pharmaceutically acceptable carrier.

19. The method or composition for use of claim 18, wherein the pharmaceutically acceptable carrier comprises water.

20. The method or composition for use of claim 18 or 19, wherein the pharmaceutically acceptable carrier comprises polysorbate 80.

21. The method or composition for use of any one of claims 18-20, wherein the pharmaceutically acceptable carrier comprises a buffer, optionally wherein the buffer is phosphate buffer and/or the pH of the formulation is about 7.0-7.5 or about 7.2.
